(19) United States
(12) Patent Application Publication
(10) Pub. No.: US 2004/0073027 A1
(43) Pub. Date: Apr. 15, 2004

(54) Method for purifying by-product gases from a melamine manufacturing installation

(76) Inventors: Gerhard Coufal, Leonding (AT); Hans Christian Wagner, Wien (AT); Wolfgang Ruech, Taiskirchen (AT)

Correspondence Address: CHRISTIE, PARKER & HALE, LLP, 350 WEST COLORADO BOULEVARD, SUITE 500, PASADENA, CA, 91105, US

(21) Appl. No.: 10/451,081

(22) PCT Filed: Dec. 19, 2001
(86) PCT No.: PCT/EP01/15058

(30) Foreign Application Priority Data
Dec. 27, 2000 (AT) A21462000

(51) Int. Cl. C07D 251/60; B01D 53/14; B01D 59/26

(52) U.S. Cl. 544/201; 95/149

(57) ABSTRACT

The invention relates to a method for purifying the by-product gases that originate in a high-pressure melamine manufacturing installation in a urea wet scrubber. According to said method, CO₂ is added to the by-product gases before or during their entry into said urea wet scrubber.

The application relates to a process for cleaning offgases from a high-pressure melamine plant by adding CO₂.

In the high-pressure process for preparing melamine, urea melt and gaseous NH₃ are reacted without the presence of a catalyst, usually at temperatures between 325 and 450° C., preferably between 350 and 425° C., and pressures between 50 and 250 bar, to give liquid melamine and offgas consisting mainly of NH₃ and CO₂ with small amounts of gaseous melamine. It is advantageous to recycle the NH₃ and CO₂ obtained at high pressure into a urea plant.

However, it is necessary for this purpose to free the offgas of the melamine fraction, since melamine is undesired in the urea plant.

U.S. Pat. No. 3,700,672 describes a process for cleaning the offgases occurring in a high-pressure melamine synthesis by closely contacting the gas mixture which contains small portions of melamine with a urea melt. The melamine is absorbed by the urea melt and a certain portion of NH₃ and CO₂ is dissolved in the urea melt. The urea melt enriched in this way and preheated by the hot offgases is subsequently fed to the high-pressure melamine reactor, while the offgases freed of melamine and cooled by the urea melt are introduced into a urea plant.

The literature describes different variants for the introduction of the offgases into a urea plant and these differ mainly in the way in which the offgas is worked up before introduction, in the location at which it is introduced into the urea plant and in the type of the urea process applied. According to U.S. Pat. No. 3,492,302, the cleaned offgas stemming from the melamine plant is absorbed in an aqueous carbonate solution and introduced as such into the rector of a urea plant. It is necessary to condense the offgas with the aid of aqueous solutions, since urea reactors are generally operated at pressures of 125 bar or higher and the melamine offgases are generally obtained at lower pressures. However, it is undesired to additionally introduce water into the urea process, since this adversely affects the conversion in the urea reactor. It would therefore be most advantageous to directly introduce the melamine offgas into the high pressure section of a melamine plant, i.e. without condensation. In this case, the melamine plant, i.e. the urea scrubber also, has to be operated at a higher pressure than the high-pressure section of the urea plant. In U.S. Pat. No. 3,723,430, the melamine offgas, depending on the pressure at which is obtained in the melamine plant, is fed to the urea plant either directly (at more than 130 bar) or together with water or aqueous carbonate solution (at less than 130 bar). WO 98/08808 likewise describes the direct introduction of the offgases of a high-pressure melamine plant into the high-pressure section of a urea stripping process.

For the direct introduction of the offgases into a urea plant, a very high pressure is therefore desired in the melamine plant. A high pressure is additionally advantageous as such for the melamine plant: a high pressure in the melamine reactor, inter alia, favors the conversion of by-products to melamine in the downstream workup section.

The difficulty of satisfying the requirements of a high pressure in the melamine plant is in particular that the high pressure required for direct introduction of the offgases into a urea plant results in excessive carbamate formation in the urea scrubber of the melamine plant.

Carbamate is undesired since it is highly corrosive at relatively high temperatures. In addition, carbamate which is fed to the melamine reactor together with the urea melt increases the energy requirement in the melamine reactor, because heat has to be fed for carbamate decomposition. It was therefore the object to find a process which enables the urea scrubber of a high-pressure melamine plant to be operated under conditions which, on the one hand substantially prevent carbamate formation and, on the other hand, enable the provision of the cleaned melamine offgases at high pressure to transfer them directly into a urea plant.

Unexpectedly, this object could be achieved by adding CO₂ to the offgases coming from the high-pressure melamine plant before or on entry into the urea scrubber.

The present invention therefore provides a process for cleaning offgases stemming from a high-pressure melamine plant in a urea scrubber, which comprises adding CO₂ to the offgases before or on entry into the urea scrubber.

The addition of CO₂ enables the urea scrubber and therefore the high-pressure section of a melamine plant to be operated at high pressures without resulting in the formation of troublesome carbamate in the urea scrubber. This on the one hand enables the direct introduction of the melamine offgases into a urea plant and, on the other hand, the high pressure in the melamine plant has a positive effect on the purity of the melamine.

The offgas to be cleaned comes from a high-pressure melamine plant in which urea is converted at a temperature of 325-450° C., preferably of 350-425° C., and a pressure of 50-450 bar, preferably of 50-250 bar, particularly preferably of 70-160 bar, to liquid melamine and offgas. To avoid the formation of by-products or for better mixing in the reactor, it is customary to add excess NH₃ gas to the melamine reactor of up to 10 mol NH₃, preferably up to 5 mol of NH₃, particularly preferably of up to 2 mol of NH₃, per mole of urea.

The liquid melamine is removed from the resulting reaction mixture of liquid melamine and offgas consisting of NH₃ gas, CO₂ gas and small amounts of gaseous melamine. The offgas obtained in this way, optionally together with the offgas from further high-pressure sections of the melamine plant which likewise consist of gaseous NH₃, CO₂ and small amounts of melamine, is mixed with CO₂ and fed to the urea scrubber.

The pressure in the urea scrubber is about the same or lower than in the apparatus from which the melaminic offgas stems, and it is, for example, between 50 and 450 bar.

Since the offgases removed from the scrubber are preferably introduced without further workup into the high-pressure section of a urea plant, the pressure in the urea scrubber is preferably about the same or higher than the pressure in the high-pressure section of the urea plant, i.e. depending on the type of the urea plant, it is preferably higher than 125 bar, particularly preferably than 155 bar. However, it may also be lower than the pressure in the high-pressure section of the urea plant.

The temperature in the urea scrubber is between 135 and 250° C., preferably between 170 and 230° C. The lower temperature limit is predetermined by the crystallization point of the urea melt. On the other hand, high temperatures in a urea melt result in strongly corrosive conditions, and also an increased extent of formation of condensation products such as biuret and triuret, which have a higher viscosity than urea and can cause conveyance problems. In addition, melamine begins to sublime from temperatures of about 250° C., which reduces the efficiency of the offgas cleaning in the urea scrubber and causes undesired melamine introduction into the urea plant.

A central problem in the operation of the urea scrubber is the formation of carbamate from the NH₃ and the CO₂ of the offgas introduced from the melamine plant. The carbamate formation is dependent on the pressure and on the temperature and also on the NH₃:CO₂ molar ratio. At a given molar ratio and a given pressure, carbamate forms from NH₃ and CO₂ below a certain temperature.

Carbamate is undesired in the melamine process for two reasons: the formation of carbamate releases heat which has to be removed in the urea scrubber in addition to the heat introduced with the offgas. In contrast, additional heat is required in the melamine reactor into which the carbamate is introduced together with the urea melt from the urea scrubber, in order to decompose the carbamate back to NH₃ and CO₂. The energy feed to the melamine reactor has to be increased by the heat required for the carbamate decomposition in order not to worsen the urea conversion in the endothermic melamine formation reaction.

To avoid carbamate formation in the urea scrubber, the temperature has to be kept above the condensation temperature depending on the pressure and on the molar NH₃:CO₂ ratio. However, since this minimum temperature likewise rises with increasing pressure, the already mentioned corrosion and viscosity problems in the urea scrubber result at the pressures required for direct offgas recycling into the urea plant.

With regard to the operation of the urea scrubber, there are therefore contradictory requirements: a very high pressure for the purposes of direct introduction of the offgases into the urea plant and also a very low temperature for the purposes of a frictionless process without corrosion problems and without melamine introduction into the urea plant. In addition, there is the need to avoid carbamate formation at all costs.

These complex requirements on an integrated melamine and urea process can be fulfilled by adding CO₂ to the offgas of the melamine plant immediately before entry into the urea scrubber.

With falling NH₃:CO₂ molar ratio, the carbamate condensation temperature shifts to lower values, i.e. the addition of CO₂ to the offgas before entry to the urea scrubber allows it to be operated at lower temperatures and the required high pressures without resulting in carbamate formation.

The offgas stemming from the high-pressure section of the melamine plant typically has an NH₃:CO₂ molar ratio of 2:1 or greater. In general, the NH₃:CO₂ molar ratio is higher than 3, since not only the NH₃ formed stoichiometrically from urea is in the offgas, but also additionally NH₃ is fed to the reactor and in most cases also to the other high-pressure apparatus of the melamine plant, which is fed to the urea scrubber together with the offgas.

At a temperature in the urea scrubber of about 200° C. and a molar ratio in the offgas of, for example, 3.3 mol of NH₃/mol CO₂, virtually no carbamate is formed at a pressure of up to about 115 bar. However, this pressure is too low for direct introduction of the offgases into the high-pressure section of the urea plant.

For the direct introduction of offgas into the high-pressure section of the urea plant, a pressure of at least 130 bar is required. The maximum molar ratio for this pressure to avoid carbamate formation is below about 2.5 mol of NH₃/mol of CO₂, i.e. a certain amount of CO₂ has to be added to the offgas from the high-pressure section of the melamine plant.

The addition of CO₂ to the offgas also brings advantages when the cleaned offgases which are removed at the top of the urea scrubber are introduced into the high-pressure section of a urea plant not directly, but rather together with aqueous solutions or in condensed form. In this case, the urea scrubber and the high-pressure section of the melamine plant can be operated at lower pressure. The addition of CO₂ to the offgas in turn enables a reduction in the temperature in the urea scrubber, without resulting in the formation of carbamate.

The offgas stemming from the high-pressure section of the melamine plant is fed to the urea scrubber at a temperature of, for example, about 300-450° C., preferably of 320-425° C. Immediately before or at the same time as the entry into the urea scrubber, CO₂, preferably gaseous CO₂, is added to the offgas. The amount of CO₂ added is to be selected in such a way that it is possible subsequent to the offgas cleaning in the urea scrubber to directly introduce the offgas into the particular urea plant. This is the case, for example, at an NH₃:CO₂ molar ratio of less than 2.5, preferably less than 2, particularly preferably less than 1.9, in particular less than 1.8. If the melamine plant is to be operated at very high pressures, molar ratios of about 1.75 or less are also possible.

The CO₂ is introduced into the offgas stream before entry into the urea scrubber, for example, via one or more nozzles, ejectors or any other spray apparatus. Good mixing of the gases is achieved, for instance, by fine distribution of the CO₂ on introduction. However, CO₂ can also be added to the offgas directly into the introduction apparatus of the offgas into the urea scrubber. The introduction apparatus may be configured, for example, as one or more spray nozzles, hole distributors or any other atomization apparatus.

The pressure of the CO₂ fed is approximately equal to or higher than the pressure at the mixing point with the offgas of the melamine plant, and is, for example, between 50 and 450 bar, preferably between 50 and 250 bar, particularly preferably between 70 and 160 bar.

The temperature of the CO₂ is, for example, between about 50 and 450° C., preferably between about 100 and 150° C., particularly preferably between 100 and 120° C.

The melaminic offgas mixed with the CO₂ is fed to the urea scrubber, into which fresh urea melt at a temperature of about 135-150° C. is fed at the same time. The fresh urea melt is contacted in countercurrent with hot offgas, resulting in heat exchange, the offgas is cooled and the urea melt is preheated. The gaseous melamine contained in the offgas is absorbed by the urea melt. At the same time, a portion of NH₃ gas and CO₂ gas corresponding to the solubility equilibrium under the pressure and temperature conditions in the urea scrubber is dissolved in the urea melt. Since the solubility of the CO₂ in the urea melt is very low, the CO₂ addition upstream of the urea scrubber does not increase the CO₂ content in the urea melt, compared with melamine offgas not admixed with CO₂.

The urea melt is removed at the bottom of the urea scrubber and fed to the melamine reactor.

The heat introduced into the urea scrubber by the hot offgas and the heat released on dissolution of NH₃ gas and CO₂ gas in the urea melt can be utilized for generating steam.

The offgases freed of melamine and consisting of NH₃ gas and CO₂ gas are removed at the top of the urea scrubber and fed to a urea plant. They are preferably introduced into a urea stripping process.

In a urea stripping plant, starting products unconverted in the urea reactor are stripped off from the synthesis mixture in a downstream stripper which is operated at about the same pressure as the urea reactor, either with the aid of a stripping gas, for example NH₃ or CO₂, or else thermally. The unconverted NH₃ and CO₂ is thus for the most part recycled into the reactor via the gas phase, so that no large water additions which reduce the conversion in the urea reactor, as is the case, for example, in conventional urea plants, are necessary. A further feature relating to the heat balance is that heat is imported only in one heater, the stripper, and is recovered in the downstream carbamate condenser.

A urea stripping plant is described, for example, in Ullmann, 5^{th} Edition, Vol. A 27, 1996, p. 344-346. It consists, for example, of a urea reactor which is generally operated at pressures between 125 and 175 bar and temperatures between 160 and 220° C. A further component is, for example, a high-pressure stripper, a high-pressure carbamate condenser, optionally a prereactor, a high-pressure scrubber, and also a low-pressure decomposer-absorber stage and also an evaporator stage.

However, the urea stripping plant can also be configured as described, for example, in Ullmann, 5^{th} Ed., Vol. A 27, 1996, p. 346-348, and comprises, for example, a reactor which is operated at about 150 bar, a stripper, a high-pressure carbamate condenser, a carbamate separator, a moderate-pressure decomposer-absorber stage, a low-pressure decomposer-absorber stage and also one or more evaporator stages.

The introduction of CO₂ into the offgas stream of a melamine plant upstream of the urea scrubber brings the abovementioned advantages and, with regards to the urea synthesis, means no additional workload. In the case of an integrated plant composed of a urea plant and melamine plant, it is meaningless for the mass balance of the urea plant whether a portion of the CO₂ required in total is conducted through the urea scrubber of the melamine plant before entry into the high-pressure section of the urea plant.

The offgas freed of melamine in the urea scrubber and mixed with CO₂ gas is removed from the scrubber at a pressure of, for example, 50-450 bar, preferably 125-250 bar, particularly preferably 70-160 bar, and a temperature of 135-250° C., preferably of 170-230° C. The offgas is subsequently introduced into the high-pressure section of the urea plant, preferably directly, i.e. without further workup. However, it can also be introduced into the high-pressure section of a urea plant together with aqueous solutions or in condensed form, for example as an aqueous carbamate solution. In this case, the offgas is condensed in one or more stages with the addition of, for example, water or aqueous carbonate or carbamate solution as a condensation assistant; the heat released can be used to preheat streams obtained in the urea process and/or for vapor generation.

The offgas from the urea scrubber can be introduced at different places in the high-pressure section of a urea plant, for example it is fed to the urea reactor, the stripper, the high-pressure carbamate condenser, the prereactor or the carbamate separator. However, it can also be introduced at any other point in the urea plant from which there is recycling into the urea reactor.

The introduction may take place directly or with the aid of ejectors or pumps.

The molar NH₃:CO₂ ratio generally typical for a urea stripping process in a synthesis reactor in the optimum case is 2:1, typically at about 3:1.

The offgas from the high-pressure melamine plant fed to the high-pressure section of the urea plant preferably has a molar NH₃:CO₂ ratio of less than 2.5. The absence of NH₃ to achieve the abovementioned molar ratio is likewise introduced into the high-pressure section of the urea plant. The introduction may in turn be at any point from which there is recycling into the urea reactor. More preferably, sufficient NH₃ is added that the molar NH₃:CO₂ ratio required for a maximum urea yield in the particular urea reactor used is achieved.

What is claimed is:

1. A process for cleaning the offgases stemming from a high-pressure melamine plant, which comprises adding CO₂ to the offgases before or on entry into the urea scrubber.

2. The process of claim 1, wherein the amount of CO₂ added is such that the molar NH₃:CO₂ ratio in the offgas before or on entry into the urea scrubber is less than 2.5, preferably less than 2, particularly preferably less than 1.9, in particular less than 1.8.

3. The process of claim 1, wherein the temperature of the added CO₂ is between 50 and 450° C., preferably between 100 and 150° C., particularly preferably between 100 and 120° C.

4. The process of claim 1, wherein the pressure of the added CO₂ is approximately equal to or higher than the pressure of the offgas of the melamine plant.

5. The process of claim 1, wherein the cleaned offgases are fed to a urea plant.

6. The process of claim 1, wherein the cleaned offgases are fed directly to a urea plant without further workup.

7. The process of claim 1, wherein the purified offgases are fed to a urea stripping plant.

8. A process for combined preparation of melamine and urea, which comprises admixing the offgases resulting from a high-pressure melamine plant with CO₂, freeing them of melamine in a urea scrubber and subsequently feeding them to the high-pressure section of a urea plant, while introducing all or some of the urea prepared there into the urea scrubber for preheating and subsequently into a high-pressure reactor for preparing melamine.
